SEQUENCE LISTING (49 sequences)

SEQ ID NO: 1 - DNA, 53 bp, Artificial Sequence
  Oligonucleotide
aaaaaagcttataattatccttagtactcgctaaagtgcgcccagatagggtg

SEQ ID NO: 2 - DNA, 60 bp, Artificial Sequence
  Oligonucleotide
cagattgtacaaatgtggtgataacagataagtcgctaaaggtaacttacctttctttgt

SEQ ID NO: 3 - DNA, 49 bp, Artificial Sequence
  Oligonucleotide
tgaacgcaagtttctaatttcgattagtactcgatagaggaaagtgtct

SEQ ID NO: 4 - DNA, 53 bp, Artificial Sequence
  Oligonucleotide
aaaaaagcttataattatccttatctaacacaagcgtgcgcccagatagggtg

SEQ ID NO: 5 - DNA, 60 bp, Artificial Sequence
  Oligonucleotide
cagattgtacaaatgtggtgataacagataagtcacaagctttaacttacctttctttgt

SEQ ID NO: 6 - DNA, 49 bp, Artificial Sequence
  Oligonucleotide
tgaacgcaagtttctaatttcggttttagatcgatagaggaaagtgtct

SEQ ID NO: 7 - DNA, 53 bp, Artificial Sequence
  Oligonucleotide
aaaaaagcttataattatccttagaagacaaaagagtgcgcccagatagggtg

SEQ ID NO: 8 - DNA, 60 bp, Artificial Sequence
  Oligonucleotide
cagattgtacaaatgtggtgataacagataagtcaaaagaaataacttacctttctttgt

SEQ ID NO: 9 - DNA, 49 bp, Artificial Sequence
  Oligonucleotide
tgaacgcaagtttctaatttcgatttcttctcgatagaggaaagtgtct

SEQ ID NO: 10 - DNA, 53 bp, Artificial Sequence
  Oligonucleotide
aaaaaagcttataattatccttatgtacccaatgtgtgcgcccagatagggtg

SEQ ID NO: 11 - DNA, 60 bp, Artificial Sequence
  Oligonucleotide
cagattgtacaaatgtggtgataacagataagtccaatgttttaacttacctttctttgt

SEQ ID NO: 12 - DNA, 49 bp, Artificial Sequence
  Oligonucleotide
tgaacgcaagtttctaatttcggttatacatcgatagaggaaagtgtct

SEQ ID NO: 13 - DNA, 53 bp, Artificial Sequence
  Oligonucleotide
aaaaaagcttataattatccttacaactcaaattggtgcgcccagatagggtg

SEQ ID NO: 14 - DNA, 60 bp, Artificial Sequence
  Oligonucleotide
cagattgtacaaatgtggtgataacagataagtcaaattggttaacttacctttctttgt

SEQ ID NO: 15 - DNA, 49 bp, Artificial Sequence
  Oligonucleotide
tgaacgcaagtttctaatttcggttagttgtcgatagaggaaagtgtct

SEQ ID NO: 16 - DNA, 53 bp, Artificial Sequence
  Oligonucleotide
aaaaaagcttataattatccttatgtatcctggaagtgcgcccagatagggtg

SEQ ID NO: 17 - DNA, 60 bp, Artificial Sequence
  Oligonucleotide
cagattgtacaaatgtggtgataacagataagtcctggaacataacttacctttctttgt

SEQ ID NO: 18 - DNA, 49 bp, Artificial Sequence; identical to SEQ ID NO: 12
  Oligonucleotide

SEQ ID NO: 19 - DNA, 53 bp, Artificial Sequence
  Oligonucleotide
aaaaaagcttataattatccttacaagacgagccagtgcgcccagatagggtg

SEQ ID NO: 20 - DNA, 60 bp, Artificial Sequence
  Oligonucleotide
cagattgtacaaatgtggtgataacagataagtcgagccagttaacttacctttctttgt

SEQ ID NO: 21 - DNA, 49 bp, Artificial Sequence
  Oligonucleotide
tgaacgcaagtttctaatttcggtttcttgtcgatagaggaaagtgtct

SEQ ID NO: 22 - DNA, 53 bp, Artificial Sequence
  Oligonucleotide
aaaaaagcttataattatccttataggtcgtgtacgtgcgcccagatagggtg

SEQ ID NO: 23 - DNA, 60 bp, Artificial Sequence
  Oligonucleotide
cagattgtacaaatgtggtgataacagataagtcgtgtactataacttacctttctttgt

SEQ ID NO: 24 - DNA, 49 bp, Artificial Sequence
  Oligonucleotide
tgaacgcaagtttctaatttcggttacctatcgatagaggaaagtgtct

SEQ ID NO: 25 - DNA, 53 bp, Artificial Sequence
  Oligonucleotide
aaaaaagcttataattatccttaatatacggattagtgcgcccagatagggtg

SEQ ID NO: 26 - DNA, 60 bp, Artificial Sequence
  Oligonucleotide
cagattgtacaaatgtggtgataacagataagtcggattaaataacttacctttctttgt

SEQ ID NO: 27 - DNA, 49 bp, Artificial Sequence
  Oligonucleotide
tgaacgcaagtttctaatttcggtttatatccgatagaggaaagtgtct

SEQ ID NO: 28 - DNA, 53 bp, Artificial Sequence
  Oligonucleotide
aaaaaagcttataattatccttacttgcctctgaggtacgcccagatagggtg

SEQ ID NO: 29 - DNA, 60 bp, Artificial Sequence
  Oligonucleotide
cagattgtacaaatgtggtgataacagataagtctctgagattaacttacctttctttgt

SEQ ID NO: 30 - DNA, 49 bp, Artificial Sequence
  Oligonucleotide
tgaacgcaagtttctaatttcggttgcaagtcgatagaggaaagtgtct

SEQ ID NO: 31 - DNA, 53 bp, Artificial Sequence
  Oligonucleotide
aaaaaagcttataattatccttacccatctacacggtgcgcccagatagggtg

SEQ ID NO: 32 - DNA, 60 bp, Artificial Sequence
  Oligonucleotide
cagattgtacaaatgtggtgataacagataagtctacacgtctaacttacctttctttgt

SEQ ID NO: 33 - DNA, 49 bp, Artificial Sequence
  Oligonucleotide
tgaacgcaagtttctaatttcggttatgggtcgatagaggaaagtgtct

SEQ ID NO: 34 - DNA, 53 bp, Artificial Sequence
  Oligonucleotide
aaaaaagcttataattatccttaattaccatccttgtgcgcccagatcgggtg

SEQ ID NO: 35 - DNA, 60 bp, Artificial Sequence
  Oligonucleotide
cagattgtacaaatgtggtgataacagataagtcatccttgataacttacctttctttgt

SEQ ID NO: 36 - DNA, 49 bp, Artificial Sequence
  Oligonucleotide
tgaacgcaagtttctaatttcggttgtaatccgatagaggaaagtgtct

SEQ ID NO: 37 - DNA, 53 bp, Artificial Sequence
  Oligonucleotide
aaaaaagcttataattatccttatatctcctggtagtgcgcccagatagggtg

SEQ ID NO: 38 - DNA, 60 bp, Artificial Sequence
  Oligonucleotide
cagattgtacaaatgtggtgataacagataagtcctggtaaataacttacctttctttgt

SEQ ID NO: 39 - DNA, 49 bp, Artificial Sequence
  Oligonucleotide
tgaacgcaagtttctaatttcggttagatatcgatagaggaaagtgtct

SEQ ID NO: 40 - DNA, 46 bp, Artificial Sequence
  Oligonucleotide
aaataatggatccagagacatcactaaaaacagaaacatatgctag

SEQ ID NO: 41 - DNA, 33 bp, Artificial Sequence
  Oligonucleotide
gaaccttctgcacttatatccattggtattccc

SEQ ID NO: 42 - DNA, 26 bp, Artificial Sequence
  Oligonucleotide
tagtaaaagggagtgtacgaccagtg

SEQ ID NO: 43 - DNA, 25 bp, Artificial Sequence
  Oligonucleotide
cgatttcgtcatggtctccaacaag

SEQ ID NO: 44 - DNA, 33 bp, Artificial Sequence
  Oligonucleotide
gcttcatatataggcagcaacccagatactggc

SEQ ID NO: 45 - DNA, 29 bp, Artificial Sequence
  Oligonucleotide
ccacccataccagagagcatttttccagg

SEQ ID NO: 46 - DNA, 35 bp, Artificial Sequence
  Oligonucleotide
ggaacctctataatatccttcactccgttaattcc

SEQ ID NO: 47 - DNA, 31 bp, Artificial Sequence
  Oligonucleotide
ggatttgttggttcttctacagtatttgcgc

SEQ ID NO: 48 - DNA, 24 bp, Artificial Sequence
  Oligonucleotide
ggtattgaatattcgaaatcaacc

SEQ ID NO: 49 - DNA, 26 bp, Artificial Sequence
  Oligonucleotide
gctgataaatatgtagctgaacctgg

(19) United States
(12) Patent Application Publication
(10) Pub. No.: US 2010/0330636 A1
(43) Pub. Date: Dec. 30, 2010

(54) PROCESS FOR THE BIOLOGICAL PRODUCTION OF N-BUTANOL WITH HIGH YIELD

(75) Inventor: Philippe SOUCAILLE, Deyme (FR)

Correspondence Address: Baker Donelson Bearman, Caldwell & Berkowitz, PC, 920 Massachusetts Ave, NW, Suite 900, Washington, DC, 20001, US

(73) Assignee: METABOLIC EXPLORER, Saint Beauzire (FR)

(21) Appl. No.: 12/823,706

(22) Filed: Jun. 25, 2010

Related U.S. Application Data
(60) Provisional application No. 61/220,596, filed on Jun. 26, 2009.

(51) Int. Cl. C12P 7/16; C12N 1/21

(52) U.S. Cl. 435/160; 435/252.7

(57) ABSTRACT

The present invention provides a method for the biological production of n-butanol at high yield from a fermentable carbon source. In one aspect of the present invention, a process for the conversion of glucose to n-butanol is achieved by the use of a recombinant organism comprising a host C. acetobutylicum transformed i) to eliminate the acetate pathway ii) to eliminate the butyrate pathway iii) to eliminate the lactate pathway and iv) to eliminate the acetone pathway. In another aspect of the present invention, the hydrogen flux is decreased and the reducing power redirected to n-butanol production by interrupting the expression of the hydrogenase gene. Optionally the n-butanol produced can be eliminated during the fermentation by gas striping and further purified by distillation.

This application claims the benefit of U.S. Provisional Application No. 61/220,596 filed Jun. 26, 2009, the entire contents of which are hereby incorporated by reference in their entirety.

### FIELD OF INVENTION

The invention comprises a process for the bioconversion of a fermentable carbon source to n-butanol at high yield by a metabolically engineered microorganism.

### BACKGROUND OF THE INVENTION

n-Butanol is a colorless, neutral liquid of medium volatility with restricted miscibility (about 7-8%) in water, but freely miscible with all common solvents such as glycols, ketones, alcohol, aldehydes, ethers, and aromatic and aliphatic hydrocarbons. n-Butanol is used i) to make other chemicals, ii) as a solvent and iii) as an ingredient in formulated products such as cosmetics. The major uses of n-butanol as a feed-stock are in the synthesis of acrylate/methacrylate esters, glycol ethers, n-Butyl acetate, amino resins and n-Butylamines. Currently, more than 9 millions tons of n-Butanol are consumed annually in the world.

More recently it has been shown that n-butanol is a better biofuel than ethanol due to lower vapour pressure, higher energy content (closer to that of gasoline) and lesser susceptibility to separation in the presence of water. Furthermore, n-butanol can be blended at higher concentrations than ethanol for use in standard vehicle engines and it does not require automakers to compromise on performance to meet environmental regulations. It is also suitable for transport in pipelines and as a result it has the potential to be introduced into gasoline quickly and avoid the need for additional large-scale supply infrastructures.

n-butanol can be produced as an acetone/n-butanol/ethanol (ABE) mixture by the fermentation of carbohydrate by solventogenic Clostridia. The ABE fermentations are biphasic. During the first acidogenic phase, high growth rate is accompanied by acetic and butyric acids production. In the second solventogenic phase growth rate decrease and the solvents (ABE) are produced with the concomitant consumption of the organic acids produced in the first phase. Carbon dioxide and hydrogen are produced throughout the fermentation.

The biological production of n-butanol requires the formation of butyryl-CoA as an intermediate which can be reduced, depending on the physiological conditions, by two different bi-functional aldehyde-alcohol dehydrogenases encoded by adhE1 and adhE2. Butyryl-CoA can also be converted to butyric acid by a phospho-transbutyrylase and a butyrate kinase encoded respectively by the ptb and buk genes. Acetate is produced from acetyl-CoA by a phosphotransacetylase encoded by the gene pta and by an acetate kinase encoded by the gene ack. Acetone is produced from aceto-acetyl-CoA (an intermediate in the production of butyryl-CoA) by a CoA-transferase and an acetoacetate decarboxylase encoded respectively by the ctfAB and adc genes. Hydrogen is produced by an iron only hydrogenase encoded by the hydA gene. When cultures are performed in the presence of carbon monoxide, a hydrogenase inhibitor, n-butanol, ethanol and lactate are the main fermentation products. Lactate is produced from pyruvate by a lactate dehydrogenase encoded by the ldh gene.

Clostridium acetobutylicum strains with an inactivated buk gene (obtained by single crossing over with a non-replicable plasmid) have already been described in the article (Green et al., 1996). The non-replicable vector pJC4BK, with a 0.8 kb internal buk fragment was integrated into the chromosomal buk gene which leaded to an inactivation of the endogenous gene. The obtained strain was named “mutant PJC4BK” from the name of the plasmid. As stated in this article, this gene integration did not completely eliminate enzyme activity or butyrate formation due to the instability of this type of gene inactivation that can reverse to wild type by plasmid excision. This mutant strain was then used in several studies (Green and Bennett, 1998; Desai and Harris, 1999; Harris et al., 2000).

Traditionally, the commercial ABE fermentation was conducted only in a batch mode due to continuous cultures instability of the producing Clostridia. Several solvent yielding fermentation processes have been described. These processes yield n-butanol, acetone and ethanol in a ratio of 6:3:1. Solvent yields of 29-34% (18-25% for n-butanol only) of fermentable carbon source have been reported in the literature. A total solvent concentration of 16-24 g/l and an n-butanol concentration of 10-14 g/l is generally the limit due to toxicity of n-butanol produced. However, these low titers of solvent no longer seem to be an economical limitation to the process as it has recently been demonstrated that solvents can be recovered during fermentation by the use of the “low cost” gas striping technology.

The problem to be solved by the present invention is to obtain a stable mutant strain with no acetate formation that could be cultured for several generations without any possibility of reversion to the wild type genotype. This strain would be useful for the biological production of n-butanol at high yield by genetically stable cultures of Clostridia, from an inexpensive carbon substrate such as glucose or other sugars. The number of biochemical steps to inactivate and the complexity of the regulation of the metabolism necessitate the use of a metabolically engineered whole cell catalyst, for an industrial feasible process of n-butanol production.

### SUMMARY OF THE INVENTION

Applicants have solved the stated problem and the present invention provides a method for the bioconvertion of a fermentable carbon source to n-butanol as a major product by genetically stable cultures of Clostridia. Glucose is used as a model substrate and recombinant Clostridium acetobutylicum is used as the model host. In one aspect of this invention, a stable recombinant C. acetobutylicum unable to produce acetate is constructed by interrupting the genes coding for the phosphotransacetylase and/or acetate kinase (pta and ack). Furthermore, a recombinant C. acetobutylicum unable to metabolize butyryl coA into butyrate is constructed by interrupting the gene coding for the butyrate kinase (buk) and/or the gene ptb. In another aspect of this invention, a recombinant C. acetobutylicum unable to produce acetone is constructed by interrupting the genes coding for the CoA-transferase (ctfAB) and/or for aceto-acetate decarboxylase (adc). In a further aspect of this invention a recombinant strain unable to produce lactate is constructed by interrupting the gene coding for the lactate dehydrogenase (ldh). In a final aspect of this invention, the flux of hydrogen production is decreased and then the flux of reducing equivalent redirected toward n-butanol production by the interruption the gene encoding the hydrogenase hydA.

The present invention may be generally applied to include any carbon substrate that is readily converted to acetyl-coA.

Accordingly it is an object of the present invention to provide a recombinant organism, useful for the production of n-butanol comprising: (a) at least an interruption of one of the two genes involved in the acetate formation, (b) at least an interruption of one of the two genes involved in the conversion of butyryl-CoA to butyrate and (c) at least an interruption of one of the two genes encoding the CoA-transferase activity. Optionally the recombinant organism may comprise i) inactivating mutations in endogenous genes selected from the group consisting of: (a) a gene encoding a polypeptide having lactate dehydrogenase activity (b) attenuation in a gene encoding a polypeptide having hydrogenase activity.

In another embodiment the invention provides a stable process for the production of n-butanol at high yield from a recombinant organism comprising: (a) contacting the recombinant organism of the present invention with at least one carbon source selected from the group consisting of monosaccharides, oligosaccharides, polysaccharides, and single-carbon substrates whereby n-butanol is produced; optionally (b) recovering the n-butanol during the production and (c) purifying n-butanol from the condensate by distillation.

### DETAILED DESCRIPTION OF THE INVENTION

The invention is related to a method for the production of n-butanol by culturing a microorganism in an appropriate culture medium comprising a source of carbon, and recovery of n-butanol from the culture medium, wherein at least one gene involved in acetate formation is interrupted in the microorganism by stable insertion of a foreign DNA into said at least one gene.

As used herein the following terms may be used for interpretation of the claims and specification.

The term “microorganism” refers to all kind of unicellular organisms, including prokaryotic organisms like bacteria, and eukaryotic organisms like yeasts. Preferentially, the microorganism is selected among Enterobacteriaceae, Bacillaceae, Streptomycetaceae, Corynebacteriaceae and Clostridiaceae. More preferentially, the microorganism is a species of Escherichia, Klebsiella, Pantoea, Salmonella Corynebacterium or Clostridium. Even more preferentially, the microorganism is a Clostridium acetobutylicum.

The expression “appropriate culture medium” refers to a culture medium adapted for the used microorganism as it is well known by the man skilled in the art.

The term “carbon substrate” or “source of carbon” means any carbon source capable of being metabolized by a microorganism wherein the substrate contains at least one carbon atom. Authors refer particularly to renewable, inexpensive and fermentable carbon sources such as monosaccharides, oligosaccharides, polysaccharides, single-carbon substrates, and polyols such as glycerol. Single carbon substrates are defined as carbon molecules that contain only one carbon atom such as methanol.

Monosaccharides of the formula (CH₂O)ₙ are also called oses or “simple sugars”; monosaccharides include saccharose, fructose, glucose, galactose and mannose.

Other carbon sources comprising more than one monosaccharide are called disaccharides, trisaccharides, oligosaccharides and polysaccharides. Disaccharides include saccharose (sucrose), lactose and maltose. Starch and hemicellulose are polysaccharides, also known as “complex sugars”.

Therefore, the term “source of carbon” means any product cited above, and mixture thereof.

The term “interruption” refers to the stable insertion of a foreign DNA into the gene, leading to the inactivation of the protein production, product of the gene. The foreign DNA is preferably an intron of a size appropriate to provide stabilization of the inactivation over the successive generations.

This technology is described precisely in patent applications EP 0 851 940 and EP 0 941 338, and has been used by the applicant as shown in the following examples. The term “Intron II” as used in the examples designates sequences of 950 bp of bacterial mobile group II intron, originating from the Lactococcus lactis L1.LtrB intron.

In the description of the present invention, enzymes are identified by their specific activities. This definition thus includes all polypeptides that have the defined specific activity also present in other organisms, more particularly in other microorganisms. Often enzymes with similar activities can be identified by their grouping to certain families defined as PFAM or COG.

PFAM (protein families database of alignments and hidden Markov models; http://www.sanger.ac.uk/Software/Pfam/) represents a large collection of protein sequence alignments. Each PFAM makes it possible to visualize multiple alignments, see protein domains, evaluate distribution among organisms, gain access to other databases, and visualize known protein structures.

COGs (clusters of orthologous groups of proteins; http://www.ncbi.nlm.nih.qov/COG/) are obtained by comparing protein sequences from 43 fully sequenced genomes representing 30 major phylogenic lines. Each COG is defined from at least three lines, which permits the identification of former conserved domains.

The means of identifying homologous sequences and their percentage homologies are well known to those skilled in the art, and include in particular the BLAST programs, which can be used from the website http://www.ncbi.nlm.nih.gov/BLAST/ with the default parameters indicated on that website. The sequences obtained can then be exploited (e.g., aligned) using, for example, the programs CLUSTALW (http://www.ebi.ac.uk/clustalw/) or MULTALIN (http://prodes.toulouse.inra.fr/multalin/cgi-bin/multalin.pl), with the default parameters indicated on those websites.

Using the references given on GenBank for known genes, those skilled in the art are able to determine the equivalent genes in other organisms, bacterial strains, yeasts, fungi, mammals, plants, etc. This routine work is advantageously done using consensus sequences that can be determined by carrying out sequence alignments with genes derived from other microorganisms, and designing degenerate probes to clone the corresponding gene in another organism. These routine methods of molecular biology are well known to those skilled in the art, and are described, for example, in Sambrook et al. (Molecular Cloning: a Laboratory Manual. 2^{nd} ed. Cold Spring Harbor Lab., Cold Spring Harbor, N.Y., 1989.).

The present invention provides a method for the fermentative batch or continuous production of n-butanol by culturing a microorganism in an appropriate culture medium comprising a carbon source and the recovery of n-butanol from the culture medium wherein at least one gene involved in acetate formation is deleted in the microorganism. Preferentially, the recovery of butanol is simultaneous to the culture.

A specific embodiment of the invention provides a method wherein the microorganism is modified to be unable to produce acetate due to the interruption of at least one gene encoding for phospho-transacetylase (pta) or acetate kinase (ack).

Interruption of genes in Clostridia can be done using the method described in patent applications EP 0 851 940 and EP 0 941 338.

In another embodiment of the invention, the microorganism is modified to be unable to produce butyrate. In a preferred embodiment, the microorganism is unable to convert butyryl-CoA to butyrate due to the interruption of at least one gene involved in butyrate formation, in particular a gene selected from the following: genes encoding for phospho-transbutyrylase (ptb) or butyrate kinase (buk).

In another embodiment of the invention, the microorganism is unable to produce acetone. Preferentially in said microorganism, at least one gene involved in acetone formation is interrupted in the microorganism.

In a preferred embodiment, this inability to produce acetone is due to an interruption of at least one of the gene encoding for CoA-transferase (ctfAB) or acetoacetate decarboxylase (adc).

In a further embodiment of the invention, the microorganism used in the method of the invention is unable to produce lactate. In particular this can be due to an interruption of the gene ldh encoding for lactate dehydrogenase.

An embodiment of the invention also provides a microorganism as described above, with a decreased flux of hydrogen production and then a redirection of the flux of reducing equivalent toward n-butanol production; this can be done by interrupting the gene encoding the hydrogenase (hydA), an enzyme that provides a sink for reducing equivalent in the form of hydrogen production.

According to the invention, the interruption of genes is performed by insertion of an intron into the gene.

Preferably, the used microorganism is selected among the group consisting of C. acetobutylicum, C. beijerinckii, C. saccharoperbutylacetonicum or C. saccharobutylicum.

In another embodiment of the invention, the culture is continuous and stable.

In another embodiment, the method according to the invention comprises the following steps:
(a) Fermentation of a microorganism producing butanol, by contacting said microorganism with at least one carbon source selected from the group consisting of glucose, xylose, arabinose, sucrose, monosaccharides, oligosaccharides, polysaccharides, cellulose, xylan, starch or its derivatives and glycerol,
(b) Optionally, recovering the n-butanol during the fermentation and
(c) Isolation of n-butanol from the condensate by distillation.

Those skilled in the art are able to define the culture conditions for the microorganisms according to the invention. In particular the clostridia are fermented at a temperature between 20° C. and 55° C., preferentially between 25° C. and 40° C., and more specifically about 35° C. for C. acetobutylicum.

The fermentation is generally conducted in fermentors with an inorganic culture medium of known defined composition adapted to the bacteria used, containing at least one simple carbon source, and if necessary a co-substrate necessary for the production of the metabolite.

The invention is also related to the microorganism as described previously. Preferably, this microorganism is selected among the group consisting of C. acetobutylicum, C. beijerinckii, C. saccharoperbutylacetonicum or C. saccharobutylicum.

### DESCRIPTION OF DRAWINGS

FIG. 1: Agarose gel of the interruption of the pta gene with an intron in sens orientation. Line 1, molecular marker; line 2, pta amplification in the wild type strain (603 pb); line 3, pta gene interrupted in sens orientation by the Intron (1552 pb).

FIG. 2: Agarose gel of the interruption of the ptb gene with an intron in sens orientation. Line 1, molecular marker; line 2, ptb gene interrupted in sens orientation by the Intron (1588 pb); line 3, empty lane; line 4, ptb amplification in the wild type strain (639 pb); line 5, molecular marker. The sizes of the molecular marker bands are indicated.

FIG. 3: Agarose gel of the interruption of the hydA gene with an intron in sens orientation. Line 1, hydA gene interrupted in sens orientation by the Intron (1438 pb); line 2, hydA amplification in the wild type strain (488 pb); line 3, molecular marker. The sizes of the molecular marker bands are indicated.

### EXAMPLES

### Example 1

### Construction of the pCONS::upp-intron Vector

This plasmid contains a pIM13 origin of replication functional in Clostridia, a catP gene conferring resistance to thiamphenicol, the upp gene and LtrA ORF required for functional expression of the group II intron RNP. In order to construct the pCONS-intron vector, we sub-cloned the sequence of the LtrA ORF into the pCONS::upp vector. The LtrA ORF region was obtained from restriction digestion of pACD4 vector (Sigma TargeTron) with XbaI and PshAI. The pSOS95 vector was digested with BamHI and SfoI blunt ended to remove the acetone formation genes while leaving the thiolase promoter region. The LtrA ORF digest product and the linearized pSOS95 vector were ligated to create the pSOS-intron vector. The pSOS-intron vector was digested by NsiI and SapI to remove the thiolase promoter and LtrA ORF. The pCONS::upp was digested with SapI and blunt ended. These fragments were ligated together to generate the pCONS::upp-intron vector.

### Example 2

### Construction of the pCONS::upp-intron ack Sense and pCONS::upp-intron ack Antisense Vectors

To inactivate the ack gene, a strain with the insertion of sense or antisense intron II in the ack gene was constructed as follows. First, a computer algorithm was used to identify target sites in the ack gene. Second, the computer algorithm outputs primer sequences (Table 1) which are used to mutate (re-target) the ack sense intron by PCR with the primers ACK 1, ACK 2, ACK 3 and the EBS universal primer or the ack antisense intron by PCR with the primers ACK 4, ACK 5, ACK 6 and the EBS universal primer. Next, the mutated 350 pb PCR fragment ack sense or antisense intron and the pCONS::upp-intron vector were digested by BsrGI and HindIII and then ligated to yield the pCONS::upp-intron ack sense or the pCONS::upp-intron ack antisense.

TABLE 1
primers sequences

| Name | | Primer sequences |
|---|---|---|
| ACK 1 | SEQ ID N^{o} 1 | AAAAAAGCTTATAATTATCCTTAGTACTCG |
| (IBS) | | CTAAAGTGCGCCCAGATAGGGTG |
| ACK 2 | SEQ ID N^{o} 2 | CAGATTGTACAAATGTGGTGATAACAGATA |
| (EBS1d) | | AGTCGCTAAAGGTAACTTACCTTTCTTTGT |
| ACK 3 | SEQ ID N^{o} 3 | TGAACGCAAGTTTCTAATTTCGATTAGTAC |
| (EBS2) | | TCGATAGAGGAAAGTGTCT |
| ACK 4 | SEQ ID N^{o} 4 | AAAAAAGCTTATAATTATCCTTATCTAACA |
| (IBS) | | CAAGCGTGCGCCCAGATAGGGTG |
| ACK 5 | SEQ ID N^{o} 5 | CAGATTGTACAAATGTGGTGATAACAGATA |
| (EBS1d) | | AGTCACAAGCTTTAACTTACCTTTCTTTGT |
| ACK 6 | SEQ ID N^{o} 6 | TGAACGCAAGTTTCTAATTTCGGTTTTAGA |
| (EBS2) | | TCGATAGAGGAAAGTGTCT |

### Example 3

### Construction of the pCONS::upp-intron pta Sense and pCONS::upp-intron pta Antisense Vectors

To inactivate the pta gene, a strain with the insertion of sense or antisense intron II in the pta gene was constructed as follows. First, a computer algorithm was used to identify target sites in the pta gene. Second, the computer algorithm outputs primer sequences (Table 2) which are used to mutate (re-target) the pta sense intron by PCR with the primers PTA 1, PTA 2, PTA 3 and the EBS universal primer or the pta antisense intron by PCR with the primers PTA 4, PTA 5, PTA 6 and the EBS universal primer. Next, the mutated 350 pb PCR fragment pta sense or antisense intron and the pCONS::upp-intron vector were digested by BsrGI and HindIII and then ligated to yield the pCONS::upp-intron pta sense or the pCONS::upp-intron pta antisense.

TABLE 2
primers sequences

| Name | | Primer sequences |
|---|---|---|
| PTA 1 | SEQ ID N^{o} 7 | AAAAAAGCTTATAATTATCCTTAGAAGAC |
| (IBS) | | AAAAGAGTGCGCCCAGATAGGGTG |
| PTA 2 | SEQ ID N^{o} 8 | CAGATTGTACAAATGTGGTGATAACAGAT |
| (EBS1d) | | AAGTCAAAAGAAATAACTTACCTTTCTTT |
| | | GT |
| PTA 3 | SEQ ID N^{o} 9 | TGAACGCAAGTTTCTAATTTCGATTTCTT |
| (EBS2) | | CTCGATAGAGGAAAGTGTCT |
| PTA 4 | SEQ ID N^{o} 10 | AAAAAAGCTTATAATTATCCTTATGTACC |
| (IBS) | | CAATGTGTGCGCCCAGATAGGGTG |
| PTA 5 | SEQ ID N^{o} 11 | CAGATTGTACAAATGTGGTGATAACAGAT |
| (EBS1d) | | AAGTCCAATGTTTTAACTTACCTTTCTTT |
| | | GT |
| PTA 6 | SEQ ID N^{o} 12 | TGAACGCAAGTTTCTAATTTCGGTTATAC |
| (EBS2) | | ATCGATAGAGGAAAGTGTCT |

### Example 4

### Construction of the pCONS::upp-intron buk Sense and pCONS::upp-intron buk Antisense Vectors

To inactivate the buk gene, a strain with the insertion of sense or antisense intron II in the buk gene was constructed as follows. First, a computer algorithm was used to identify target sites in the buk gene. Second, the computer algorithm outputs primer sequences (Table 3) which are used to mutate (re-target) the buk sense intron by PCR with the primers BUK 1, BUK 2, BUK 3 and the EBS universal primer or the buk antisense intron by PCR with the primers BUK 4, BUK 5, BUK 6 and the EBS universal primer. Next, the mutated 350 pb PCR fragment buk sense or antisense intron and the pCONS::upp-intron vector were digested by BsrGI and HindIII and then ligated to yield the pCONS::upp-intron buk sense or the pCONS::upp-intron buk antisense.

TABLE 3
primers sequences

| Name | | Primer sequences |
|---|---|---|
| BUK 1 | SEQ ID N^{o} 13 | AAAAAAGCTTATAATTATCCTTACAAC |
| (IBS) | | TCAAATTGGTGCGCCCAGATAGGGTG |
| BUK 2 | SEQ ID N^{o} 14 | CAGATTGTACAAATGTGGTGATAACAG |
| (EBS1d) | | ATAAGTCAAATTGGTTAACTTACCTTT |
| | | CTTTGT |
| BUK 3 | SEQ ID N^{o} 15 | TGAACGCAAGTTTCTAATTTCGGTTAG |
| (EBS2) | | TTGTCGATAGAGGAAAGTGTCT |
| BUK 4 | SEQ ID N^{o} 16 | AAAAAAGCTTATAATTATCCTTATGTA |
| (IBS) | | TCCTGGAAGTGCGCCCAGATAGGGTG |
| BUK 5 | SEQ ID N^{o} 17 | CAGATTGTACAAATGTGGTGATAACAG |
| (EBS1d) | | ATAAGTCCTGGAACATAACTTACCTTT |
| | | CTTTGT |
| BUK 6 | SEQ ID N^{o} 18 | TGAACGCAAGTTTCTAATTTCGGTTAT |
| (EBS2) | | ACATCGATAGAGGAAAGTGTCT |

### Example 5

### Construction of the pCONS::upp-intron ptb Sense Vector

To inactivate the ptb gene, a strain with the insertion of sense intron II in the ptb gene was constructed as follows. First, a computer algorithm was used to identify target sites in the ptb gene. Second, the computer algorithm outputs primer sequences (Table 4) which are used to mutate (re-target) the ptb sense intron by PCR with the primers PTB 1, PTB 2, PTB 3 and the EBS universal primer. Next, the mutated 350 pb PCR fragment ptb sense intron and the pCONS::upp-intron vector were digested by BsrGI and HindIII and then ligated to yield the pCONS::upp-intron ptb sense.

TABLE 4
primers sequences

| Name | | Primer sequences |
|---|---|---|
| PTB 1 | SEQ ID N^{o} 19 | AAAAAAGCTTATAATTATCCTTACAAGA |
| (IBS) | | CGAGCCAGTGCGCCCAGATAGGGTG |
| PTB 2 | SEQ ID N^{o} 20 | CAGATTGTACAAATGTGGTGATAACAGA |
| (EBS1d) | | TAAGTCGAGCCAGTTAACTTACCTTTCT |
| | | TTGT |
| PTB 3 | SEQ ID N^{o} 21 | TGAACGCAAGTTTCTAATTTCGGTTTCT |
| (EBS2) | | TGTCGATAGAGGAAAGTGTCT |

### Example 6

### Construction of the pCONS::upp-intron ctfA Sense and pCONS::upp-intron ctfA Antisense Vectors

To inactivate the ctfA gene, a strain with the insertion of sense or antisense intron II in the ctfA gene was constructed as follows. First, a computer algorithm was used to identify target sites in the ctfA gene. Second, the computer algorithm outputs primer sequences (Table 5) which are used to mutate (re-target) the ctfA sense intron by PCR with the primers CTFA 1, CTFA 2, CTFA 3 and the EBS universal primer or the ctfA antisense intron by PCR with the primers CTFA 4, CTFA 5, CTFA 6 and the EBS universal primer. Next, the mutated 350 pb PCR fragment ctfA sense or antisense intron and the pCONS::upp-intron vector were digested by BsrGI and HindIII and then ligated to yield the pCONS::upp-intron ctfA sense or the pCONS::upp-intron ctfA antisense.

TABLE 5
primers sequences

| Name | | Primer sequences |
|---|---|---|
| CTFA 1 | SEQ ID N^{o} 22 | AAAAAAGCTTATAATTATCCTTATAGGT |
| (IBS) | | CGTGTACGTGCGCCCAGATAGGGTG |
| CTFA 2 | SEQ ID N^{o} 23 | CAGATTGTACAAATGTGGTGATAACAGA |
| (EBS1d) | | TAAGTCGTGTACTATAACTTACCTTTCT |
| | | TTGT |
| CTFA 3 | SEQ ID N^{o} 24 | TGAACGCAAGTTTCTAATTTCGGTTACC |
| (EBS2) | | TATCGATAGAGGAAAGTGTCT |
| CTFA 4 | SEQ ID N^{o} 25 | AAAAAAGCTTATAATTATCCTTAATATA |
| (IBS) | | CGGATTAGTGCGCCCAGATAGGGTG |
| CTFA 5 | SEQ ID N^{o} 26 | CAGATTGTACAAATGTGGTGATAACAGA |
| (EBS1d) | | TAAGTCGGATTAAATAACTTACCTTTCT |
| | | TTGT |
| CTFA 6 | SEQ ID N^{o} 27 | TGAACGCAAGTTTCTAATTTCGGTTTAT |
| (EBS2) | | ATCCGATAGAGGAAAGTGTCT |

### Example 7

### Construction of the pCONS::upp-intron ldh Sense and pCONS::upp-intron ldh Antisense Vectors

To inactivate the ldh gene, a strain with the insertion of sense or antisense intron II in the ldh gene was constructed as follows. First, a computer algorithm was used to identify target sites in the ldh gene. Second, the computer algorithm outputs primer sequences (Table 6) which are used to mutate (re-target) the ldh sense intron by PCR with the primers LDH 1, LDH 2, LDH 3 and the EBS universal primer or the ldh antisense intron by PCR with the primers LDH 4, LDH 5, LDH 6 and the EBS universal primer. Next, the mutated 350 pb PCR fragment ldh sense or antisense intron and the pCONS::upp-intron vector were digested by BsrGI and HindIII and then ligated to yield the pCONS::upp-intron ldh sense or the pCONS::upp-intron ldh antisense.

TABLE 6
primers sequences

| Name | | Primer sequences |
|---|---|---|
| LDH 1 | SEQ ID N^{o} 28 | AAAAAAGCTTATAATTATCCTTACTTGC |
| (IBS) | | CTCTGAGGTACGCCCAGATAGGGTG |
| LDH 2 | SEQ ID N^{o} 29 | CAGATTGTACAAATGTGGTGATAACAGA |
| (EBS1d) | | TAAGTCTCTGAGATTAACTTACCTTTCT |
| | | TTGT |
| LDH 3 | SEQ ID N^{o} 30 | TGAACGCAAGTTTCTAATTTCGGTTGCA |
| (EBS2) | | AGTCGATAGAGGAAAGTGTCT |
| LDH 4 | SEQ ID N^{o} 31 | AAAAAAGCTTATAATTATCCTTACCCAT |
| (IBS) | | CTACACGGTGCGCCCAGATAGGGTG |
| LDH 5 | SEQ ID N^{o} 32 | CAGATTGTACAAATGTGGTGATAACAGA |
| (EBS1d) | | TAAGTCTACACGTCTAACTTACCTTTCT |
| | | TTGT |
| LDH 6 | SEQ ID N^{o} 33 | TGAACGCAAGTTTCTAATTTCGGTTATG |
| (EBS2) | | GGTCGATAGAGGAAAGTGTCT |

### Example 8

### Construction of the pCONS::upp-intron hydA Sense and pCONS::upp-intron hydA Antisense Vectors

To inactivate the hydA gene, a strain with the insertion of sense or antisense intron II in the hydA gene was constructed as follows. First, a computer algorithm was used to identify target sites in the hydA gene. Second, the computer algorithm outputs primer sequences (Table 7) which are used to mutate (re-target) the hydA sense intron by PCR with the primers HYDA 1, HYDA 2, HYDA 3 and the EBS universal primer or the hydA antisense intron by PCR with the primers HYDA 4, HYDA 5, HYDA 6 and the EBS universal primer. Next, the mutated 350 pb PCR fragment hydA sense or antisense intron and the pCONS::upp-intron vector were digested by BsrGI and HindIII and then ligated to yield the pCONS::upp-intron hydA sense or the pCONS::upp-intron hydA antisense.

TABLE 7
primers sequences

| Name | | Primer sequences |
|---|---|---|
| HYDA 1 | SEQ ID N^{o} 34 | AAAAAAGCTTATAATTATCCTTAATTAC |
| (IBS) | | CATCCTTGTGCGCCCAGATCGGGTG |
| HYDA 2 | SEQ ID N^{o} 35 | CAGATTGTACAAATGTGGTGATAACAGA |
| (EBS1d) | | TAAGTCATCCTTGATAACTTACCTTTCT |
| | | TTGT |
| HYDA 3 | SEQ ID N^{o} 36 | TGAACGCAAGTTTCTAATTTCGGTTGTA |
| (EBS2) | | ATCCGATAGAGGAAAGTGTCT |
| HYDA 4 | SEQ ID N^{o} 37 | AAAAAAGCTTATAATTATCCTTATATCT |
| (IBS) | | CCTGGTAGTGCGCCCAGATAGGGTG |
| HYDA 5 | SEQ ID N^{o} 38 | CAGATTGTACAAATGTGGTGATAACAGA |
| (EBS1d) | | TAAGTCCTGGTAAATAACTTACCTTTCT |
| | | TTGT |
| HYDA 6 | SEQ ID N^{o} 39 | TGAACGCAAGTTTCTAATTTCGGTTAGA |
| (EBS2) | | TATCGATAGAGGAAAGTGTCT |

### Example 9

### Construction of Strains Unable to Produce Acetate

### Clostridium acetobutylicum Δcac1515 Δupp pta::intronII

The pCONS::upp-intron pta sense plasmid was used to transform by electroporation C. acetobutylicum MGC Δcac15 Δupp strain. After selection on Petri plate for clones resistant to thiamphenicol (50 μg/ml), one colony was cultured for 24 hours in liquid synthetic medium with thiamphenicol at 50 μg/ml and 100 μl of undiluted culture was plated on RCA with thiamphenicol at 50 μg/ml. The insertion of the intron II in clones resistant to thiamphenicol was checked by PCR analysis with primers PTA 7 and PTA 8 (Table 8) located outside of the pta gene.

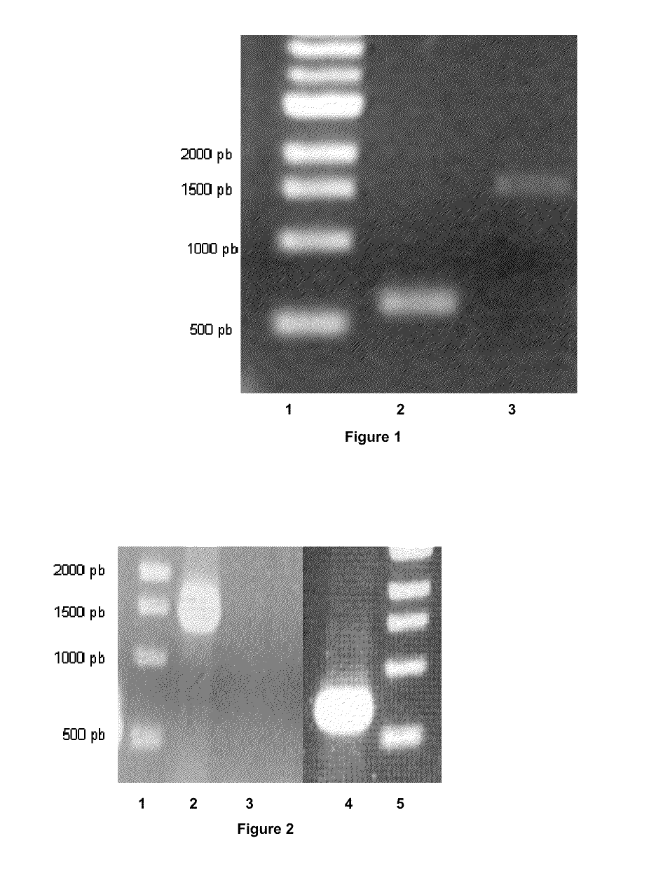

One colony of the C. acetobutylicum pta::intron II pCONS::upp-intron pta sense strain was then cultured in chemostat on synthetic medium without antibiotic. Serial dilution of the culture was regularly plated on 2YTG plates containing 5-Fluorouracyl (5-FU). Several clones were obtained which were resistant to 5-FU. When transferred on 2YTG plates plus or minus thiamphenicol, a large number of these clones did show thiamphenicol resistance, suggesting they still contained the pCONS::upp-intron pta sense with a mutation in the upp gene, conferring resistance to 5-FU. However, some clones could be shown to be thiamphenicol sensitive. We could confirm that these last clones did not contain the pCONS::upp-intron pta sense plasmid but still had the insertion of the intron II in the pta gene (FIG. 1). We could therefore easily select for a complementary mutation allowing for the inactivation of the phosphotransacetylase in this strain.

TABLE 8
primers sequences

| | | |
|---|---|---|
| PTA 7 | SEQ ID N^{o} 40 | ATTAAATGCAAACTTAAATGCAATTTTTA |
| PTA 8 | SEQ ID N^{o} 41 | GTTGTGTGAACAGCTCCTGAAACCATTCC |

### Example 10

### Construction of Strains Unable to Produce Acetate and Butyrate

### C. acetobutylicum Δcac1515 Δupp ack::intronII ptb::intronII

The pCONS::upp-intron ptb sense plasmid was used to transform by electroporation C. acetobutylicum MGC Δcac15 Δupp ack::intron II strain. After selection on Petri plate for clones resistant to thiamphenicol (50 μg/ml), one colony was cultured for 24 hours in liquid synthetic medium with thiamphenicol at 50 μg/ml and 100 μl of undiluted culture was plated on RCA with thiamphenicol at 50 μg/ml. The insertion of the intron II in clones resistant to thiamphenicol was checked by PCR analysis with primers PTB 7 and PTB 8 (Table 9) located outside of the ptb gene (FIG. 2). The C. acetobutylicum Δcac15 Δupp ack::intron II ptb::intron strain which has lost pCONS::upp-intron ptb sense was isolated on RCA with 5-FU at 400 μM.

TABLE 9
primers sequences

| | | |
|---|---|---|
| PTB 7 | SEQ ID N^{o} 42 | GTCTATCAAATTTCTCAGTTTCAAATACTG |
| PTB 8 | SEQ ID N^{o} 43 | GAAAGTATTTCACTAAAAGGACTATATCC |

### Example 11

### Construction of Strains Unable to Produce Acetate, Butyrate and Acetone

### C. acetobutylicum Δcac1515 Δupp ack::intronII ptb::intronII ctfA::intronII

The pCONS::upp-intron ctfA sense plasmid was used to transform by electroporation C. acetobutylicum MGC Δcac15 Δupp ack::intron II ptb::intron II strain. After selection on Petri plate for clones resistant to thiamphenicol (50 μg/ml), one colony was cultured for 24 hours in liquid synthetic medium with thiamphenicol at 50 μg/ml and 100 μl of undiluted culture was plated on RCA with thiamphenicol at 50 μg/ml. The insertion of the intron II in clones resistant to thiamphenicol was checked by PCR analysis with primers CTFA 7 and CTFA 8 (Table 10) located outside of the ctfA gene. The C. acetobutylicum Δcac15 Δupp ack::intron II ptb::intron II ctfA::intron II strain which has lost pCONS::upp-intron ctfA sense was isolated on RCA with 5-FU at 400 μM.

TABLE 10
primers sequences

| | | |
|---|---|---|
| CTFA 7 | SEQ ID N^{o} 44 | GCTTCATATATAGGCAGCAACCCAGATACTGGC |
| CTFA 8 | SEQ ID N^{o} 45 | CCACCCATACCAGAGAGCATTTTTCCAGG |

### Example 12

### Construction of Strains Unable to Produce Acetate, Butyrate, Acetone and Lactate

### C. acetobutylicum Δcac1515 Δupp ack::intronII ptb::intronII ctfA::intronII ldh::intronII

The pCONS::upp-intron ldh sense plasmid was used to transform by electroporation C. acetobutylicum MGC Δcac15 Δupp ack::intron II ptb::intron II ctfA::intron II strain. After selection on Petri plate for clones resistant to thiamphenicol (50 μg/ml), one colony was cultured for 24 hours in liquid synthetic medium with thiamphenicol at 50 μg/ml and 100 μl of undiluted culture was plated on RCA with thiamphenicol at 50 μg/ml. The insertion of the intron II in clones resistant to thiamphenicol was checked by PCR analysis with primers LDH 7 and LDH 8 (Table 11) located outside of the ldh gene. The C. acetobutylicum Δcac15 Δupp ack::intron II ptb::intron II ctfA::intron II ldh::intron II strain which has lost pCONS::upp-intron ldh sense was isolated on RCA with 5-FU at 400 μM.

TABLE 11
primers sequences

| | | |
|---|---|---|
| LDH 7 | SEQ ID N^{o} 46 | GGAACCTCTATAATATCCTTCACTCCGTTA |
| | | ATTCC |
| LDH 8 | SEQ ID N^{o} 47 | GGATTTGTTGGTTCTTCTACAGTATTTGCGC |

### Example 13

### Construction of Strains with Lower Hydrogen Production

### C. acetobutylicum Δcac1515 Δupp hydA::intronII

The pCONS::upp-intron hydA sense plasmid was used to transform by electroporation C. acetobutylicum MGC Δcac15 Δupp strain. After selection on Petri plate for clones resistant to thiamphenicol (50 μg/ml), one colony was cultured for 24 hours in liquid synthetic medium with thiamphenicol at 50 μg/ml and 100 μl of undiluted culture was plated on RCA with thiamphenicol at 50 μg/ml. The insertion of the intron II in clones resistant to thiamphenicol was checked by PCR analysis (with primers HYDA 7 and HYDA 8 (Table 12) located outside of the hydA gene.

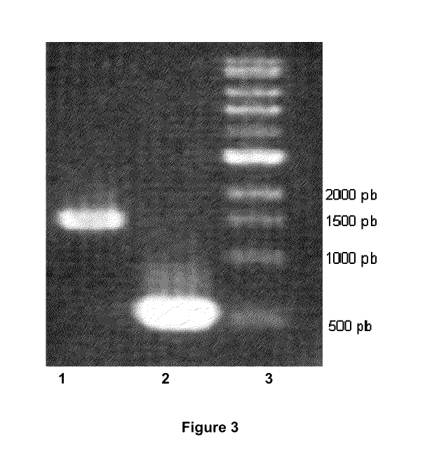

One colony of the C. acetobutylicum hydA::intron II pCONS::upp-intron hydA sense strain was then cultured in chemostat on synthetic medium without antibiotic. Serial dilution of the culture was regularly plated on 2YTG plates containing 5-Fluorouracyl (5-FU). Several clones were obtained which were resistant to 5-FU. When transferred on 2YTG plates plus or minus thiamphenicol, a large number of these clones did show thiamphenicol resistance, suggesting they still contained the pCONS::upp-intron hydA sense with a mutation in the upp gene, conferring resistance to 5-FU. However, some clones could be shown to be thiamphenicol sensitive. We could confirm that these last clones did not contain the pCONS::upp-intron hydA sense plasmid but still had the insertion of the intron II in the hydA gene (FIG. 3). We could therefore easily select for a complementary mutation allowing for the inactivation of the hydrogenase in this strain.

TABLE 12
primers sequences

| | | |
|---|---|---|
| HYDA 7 | SEQ ID N^{o} 48 | GGTATTGAATATTCGAAATCAACC |
| HYDA 8 | SEQ ID N^{o} 49 | GCTGATAAATATGTAGCTGAACCTGG |

### Example 14

### Batch Fermentation of n-butanol Producing Strains

Strains were initially analyzed in anaerobic flask cultures in the synthetic medium described by Soni et al (Soni et al, 1987, Appl. Microbiol. Biotechnol. 27:1-5) supplemented with 2.5 g/l of ammonium acetate. An overnight culture at 35° C. was used to inoculate a 30 ml culture to an OD600 of 0.05. After incubation of the culture for 3 days at 35° C., glucose, organic acids and solvents were analyzed by HPLC using a Biorad HPX 97H column for the separation and a refractometer for the detection.

Strains with the correct phenotype were subsequently tested under production conditions in 300 ml fermentors (DASGIP) using an anaerobic batch protocol.

For this purpose the fermentor was filled with 250 ml of synthetic medium, sparged with nitrogen for 30 min and inoculated with 25 ml of preculture to an optical density (OD600 nm) between 0.05 and 0.1.

The temperature of the culture was maintained constant at 35° C. and the pH was permanently adjusted at 5.5 using an NH₄OH solution. The agitation rate was maintained at 300 rpm during the fermentation.

### Example 15

### Continuous Fermentation of n-butanol Producing Strains

The best n-butanol producing strain was analyzed in chemostat cultures in the synthetic medium described by Soni et al (Soni et al, 1987, Appl. Microbiol. Biotechnol. 27:1-5). An overnight culture at 35° C. was used to inoculate a 300 ml fermentors (DASGIP) using an anaerobic chemostat protocol.

For this purpose the fermentor was filled with 250 ml of synthetic medium, sparged with nitrogen for 30 min and inoculated with 25 ml of preculture to an optical density (OD600 nm) between 0.05 and 0.1. After 12 hours of batch culture at 35° C., pH 5.5 (regulated using an NH₄OH solution) and an agitation rate of 300 rpm, the fermentor was continuously fed with oxygen free synthetic medium at a dilution rate of 0.05 h-1 while the volume was kept constant by sequential removal of fermented medium. Stability of the culture was followed by products analysis using the HPLC protocol previously described.

### REFERENCES

Desai R P, Harris L M, Welker N E, Papoutsakis E T. Metabolic flux analysis elucidates the importance of the acid-formation pathways in regulating solvent production by Clostridium acetobutylicum. Metab. Eng. 1999, 1: 206-13.
Green E M, Bennett G N. Genetic manipulation of acid and solvent formation in Clostridium acetobutylicum ATCC 824 Biotechnol. Bioeng. 1998, 58: 215-21.
Green E M, Boynton Z L, Harris L M, Rudolph F B, Papoutsakis E T, Bennett G N. Genetic manipulation of acid formation pathways by gene inactivation in Clostridium acetobutylicum ATCC 824. Microbiology. 1996, 142: 2079-86.
Harris L M, Desai R P, Welker N E, Papoutsakis E T. Characterization of recombinant strains of the Clostridium acetobutylicum butyrate kinase inactivation mutant: need for new phenomenological models for solventogenesis and butanol inhibition? Biotechnol. Bioeng. 2000, 67: 1-11.
Soni B. K., Soucaille P. Goma G. Continuous acetone butanol fermentation: influence of vitamins on the metabolic activity of Clostridium acetobutylicum. Appl. Microbiol. Biotechnol. 1987, 27: 1-5.

1) A method for the production of n-butanol by culturing a microorganism in an appropriate culture medium comprising a source of carbon and recovery of n-butanol from the culture medium, wherein at least one gene involved in acetate formation is interrupted in the microorganism by stable insertion of a foreign DNA into said at least one gene.

2) The method according to claim 1 wherein the interrupted gene is at least one of the following genes:
pta encoding phospho-transacetylase
ack encoding acetate kinase.

3) The method as claimed in claim 1 wherein the microorganism is modified to be unable to produce butyrate.

4) The method as claimed in claim 3 in which at least one gene involved in butyrate formation is interrupted.

5) The method according to claim 4 wherein the interrupted gene is selected among the following:
ptb encoding phospho-transbutyrylase
buk encoding butyrate kinase.

6) The method according to claim 1 wherein at least one gene involved in acetone formation is interrupted in the microorganism.

7) The method according to claim 6 wherein at least one of the following genes is interrupted:
ctfAB encoding CoA-transferase
adc encoding aceto-acetate decarboxylase.

8) The method according to claim 1 wherein the microorganism is modified to be unable to produce lactate.

9) The method according to claim 8 wherein the ldh gene is interrupted.

10) The method as claimed in claim 1, wherein the hydrogen flux is decreased and the reducing power redirected to butanol production.

11) The method as claimed in claim 10 wherein the hydA gene is interrupted.

12) The method as claimed in claim 1, wherein the at least one gene is interrupted by insertion of an intron into the gene.

13) The method according to claim 1 wherein the microorganism is selected among the group consisting of C. acetobutylicum, C. beijerinckii, C. saccharoperbutylacetonicum or C. saccharobutylicum.

14) The method according to claim 1 wherein the culture is continuous and stable.

15) The method according to claim 14 comprising the following steps:
a) Fermentation of the microorganism producing n-butanol;
b) Optionally the elimination of n-butanol during the fermentation;
c) Isolation of n-butanol from the condensate by distillation.

16) The microorganism as defined in claim 1.
